(19) United States
(12) Patent Application Publication
(10) Pub. No.: US 2009/0306519 A1
(43) Pub. Date: Dec. 10, 2009

(54) MEASUREMENT WITH MULTIPLEXED DETECTION

(75) Inventor: Levinus Pieter Bakker, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,047

(22) PCT Filed: Jul. 16, 2007
(86) PCT No.: PCT/IB07/52823

§ 371 Date: Jan. 26, 2009

(30) Foreign Application Priority Data
Jul. 31, 2006 (EP) 06118177.2

(51) Int. Cl. A61B 6/00

(52) U.S. Cl. 600/476

(57) ABSTRACT

The invention relates to a device (1) for imaging an interior of a turbid medium (45) comprising: a) a measurement volume (15) for receiving the turbid medium (45); b) a light source (5) for irradiating the turbid medium (45); c) a photodetector unit (10) for detecting light emanating from exit positions (60) located on a boundary of the measurement volume (15) as a result of >irradiating the turbid medium (45). The device (1) is adapted to collect light from spatially distinct beams of light emanating from a single exit position (60). The single exit position (60) may, for instance, come in the form of an optical channel (25b) or it may simply be an area of skin tissue from which light emanates. In one embodiment of the device (1) according to the invention multiple optical light guides (55) are optically coupled to the single exit position (60).

The invention relates to a device for imaging an interior of a turbid medium comprising:

a) a measurement volume for receiving the turbid medium;

b) a light source for irradiating the turbid medium;

c) a photodetector unit for detecting light emanating from exit positions located on a boundary of the measurement volume as a result of irradiating the turbid medium.

The invention also relates to a medical image acquisition device comprising:

a) a measurement volume for receiving a turbid medium;

b) a light source for irradiating the turbid medium;

c) a photodetector unit for detecting light emanating from exit positions located on a boundary of the measurement volume as result of irradiating the turbid medium.

An embodiment of a device of this kind is known from U.S. Pat. No. 6,327,488 B1. The known device can be used for imaging an interior of a turbid medium, such as biological tissues. In medical diagnostics the device may be used for imaging an interior of a female breast. The measurement volume receives a turbid medium, such as a breast. The turbid medium is then irradiated with light from the light source. Typically, light having a wavelength within the range of 400 nm to 1400 nm is used for this purpose. Light emanating from the measurement volume from exit positions as a result of irradiating the turbid medium is detected by the photodetector unit and used to derive an image of an interior of the turbid medium.

It is a drawback of the known device that the signal resulting from detecting light emanating from the measurement volume is sensitive to noise. The sensitivity to noise results from the fact that diffuse optical tomography measurements require very sensitive detection of sometimes weak light signals.

It is an object of the invention to improve the signal to noise ratio of the signal resulting from detecting light emanating from the measurement volume.

According to the invention this object is realized in that the device is arranged to collect light from spatially distinct beams of light emanating from a single exit position.

The invention is based on the recognition that light emanating from the single exit position is emitted diffusely. Hence, detecting spatially distinct light beams emanating from a single exit position is possible. Detecting spatially distinct light beams emanating from a single exit position means that multiple signals are detected for a single exit position. In this way, the number of signals obtained over a specific period of time for a single exit position, that is the number of measurements at that single exit position per unit of time, is increased. The increase in the number of measurements is used to increase signal to noise ratios. The increase in the number of measurements may further be used to reduce measurement times as a specific amount of signal may be obtained over a shorter period of time compared to the situation in the known device.

It is an additional advantage of the invention that the detected light from the spatially distinct light beams originates from a single exit position as opposed to multiple exit positions. As the light emanates from a single exit position in a diffuse manner, the spatially distinct light beams therefore have the same intensity and carry substantially the same information.

An embodiment of the device according to the invention is characterized in that the device comprises a plurality of optical light guides having entry openings for light and having acceptance angles for light to enter the entry openings for light and being arranged such that the plurality of optical light guides is optically coupled to a single exit position such that the distances between the entry openings for light of the optical light guides and the single exit position correspond to the acceptance angles of the optical light guides. The distance between an entry opening for light of an optical light guide and a single exit position correspond to each other such that the tangent of the acceptance angle for light (θ) of the optical light guide is given by the quotient of half the width (w) of the single exit position and the distance (d) between the entry opening for light of the optical light guide and the single exit position, i.e. tan(θ)=w/2d. This embodiment has the advantage that it is easy to implement in the known device, as the known device already comprises single exit positions optically coupled to single optical light guides. Arranging the device such that the distance between the entry openings for light of the optical light guides and the single exit position corresponds to the acceptance angles of the optical light guides, maximizes the amount of light exiting the single exit position that can enter the optical light guides. In the known device a receptacle bounds the measurement volume for receiving the turbid medium. Light emanating from the measurement volume exits the receptacle through a plurality of exit positions in the wall of the receptacle. A single optical light guide is directly coupled to each of these exit positions.

A further embodiment of the device according to the invention is characterized in that the device is arranged to apply at least two different detection schemes to at least two spatially distinct light beams emanating from a single exit position. This embodiment has the advantage that various types of information may be obtained simultaneously from spatially distinct light beams emanating from a single exit position. One detection scheme, for instance, may involve the use of a fluorescence filter in the path of a light beam. Another detection scheme may involve the use of no filters at all. In this way, fluorescence and transmission measurements may be performed on a turbid medium simultaneously, thus further reducing overall measurement times as compared to a situation in which both measurements are performed one after the other and reducing noise

A further embodiment of the device according to the invention is characterized in that the device comprises means for focusing at least one light beam emanating from a single exit position. Focusing means comprise a lens and a mirror any of which may be used to focus a light beam emanating from the single exit position. This embodiment has the advantage that it enables the use of an interference filter in the focused beam. Interference filters must preferably be placed in the path of a parallel light beam to function properly. Focusing means may then be used to couple light from the light beam to the photodetector unit, for instance, by using a lens to focus light of a parallel light beam emanating from the single exit position onto the entry opening for light of an optical fiber coupled to the photodetector unit.

The medical image acquisition device according to the invention is defined in claim 5. According to the invention the medical image acquisition device is arranged to collect light from spatially distinct beams of light emanating from a single exit position. If, for instance, the device is used to image an interior of a female breast, as is done in medical diagnostics, the device would benefit from any of the previous embodiments.

These and other aspects of the invention will be further elucidated and described with reference to the drawings, in which:

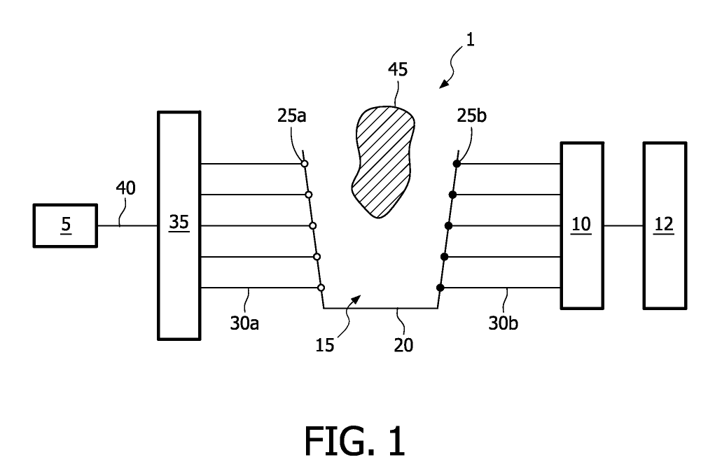

FIG. 1 schematically shows a device for imaging an interior of a turbid medium as known from prior art;

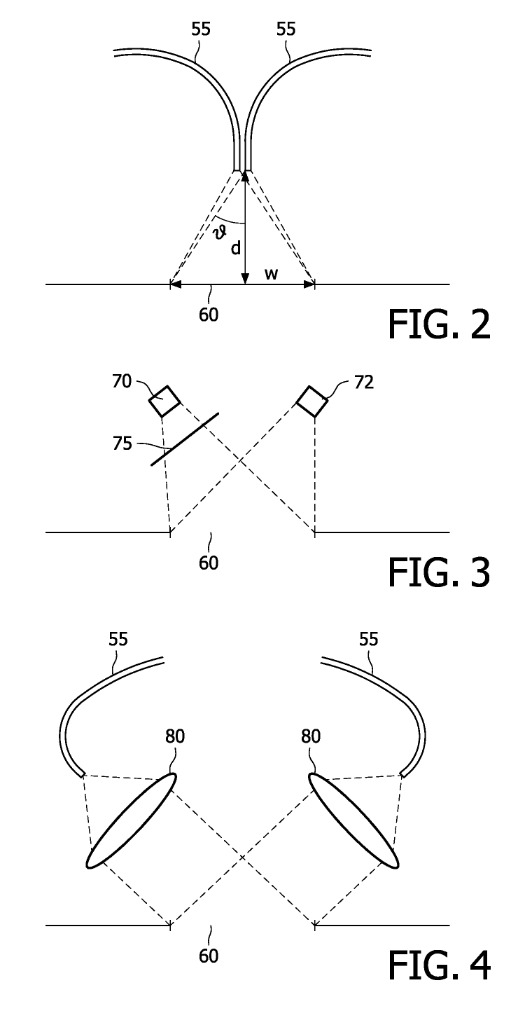

FIG. 2 schematically shows how a device may be arranged to detect light from spatially distinct beams of light emanating from a single exit position;

FIG. 3 schematically shows two different detection schemes applied to two spatially distinct light beams;

FIG. 4 schematically shows means for focusing a light beam emanating from a single exit position;

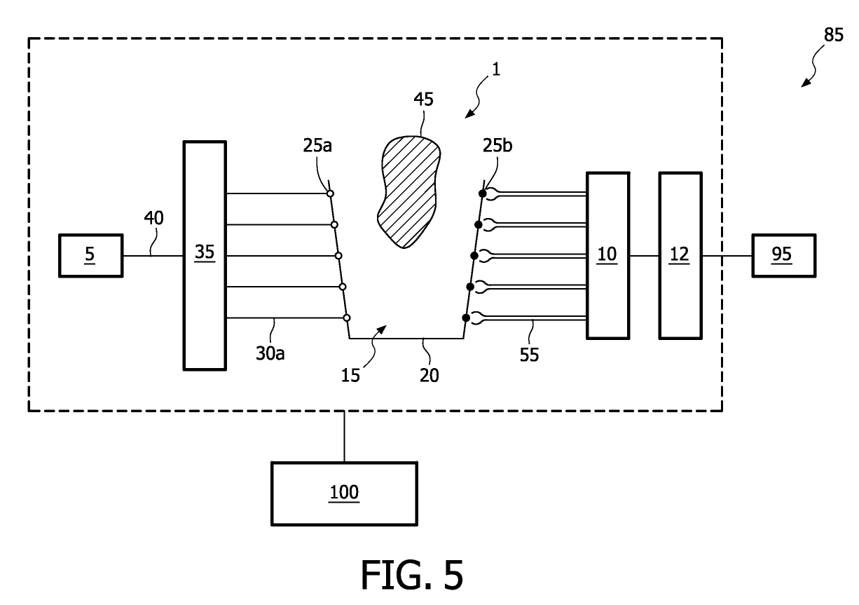

FIG. 5 shows an embodiment of a medical image acquisition device according to the invention.

FIG. 1 schematically shows a device 1 for imaging an interior of a turbid medium as known from prior art. The device 1 comprises a light source 5, a photodetector unit 10, an image reconstruction unit 12 for reconstructing an image of an interior of a turbid medium 45 on the basis of light detected by the photodetector unit 10, a measurement volume 15 bound by a receptacle 20, said receptacle comprising a plurality of optical channels 25a and 25b, and light guides 30a and 30b coupled to said optical channels. The device 1 further includes a selection unit 35 for coupling the input light guide 40 to a number of optical channels selected from the plurality of optical channels 25a in the receptacle 20. For the sake of clarity, optical channels 25a and 25b have been positioned at opposite sides of the receptacle 20. In reality, however, both types of optical channel may be distributed around the measurement volume 15. The turbid medium 45 is placed inside the measurement volume 15. The turbid medium 45 is then irradiated with light from the light source 5 from a plurality of positions by coupling the light source 5 using the selection unit 35 to successively selected optical channels 25a. The light is chosen such that it is capable of propagating through the turbid medium 45. Light emanating from the measurement volume 15 as a result of irradiating the turbid medium 45 is detected from a plurality of exit positions using optical channels 25b and using photodetector 10. The detected light is then used to derive an image of an interior of the turbid medium 45. Deriving an image of an interior of the turbid medium 45 based on the detected light is possible as at least part of this light has traveled through the turbid medium 45 and, as a consequence, contains information relating to an interior of the turbid medium 45. The light has been intentionally chosen such that it is capable of propagating through the turbid medium 45. If, as may be the case in medical diagnostics, the device 1 is used for imaging an interior of a female breast, suitable light is, for instance, laser light with a wavelength of 660 nm or laser light with a wavelength of 810 nm. In FIG. 1 the measurement volume 15 is bound by a receptacle 20. However, this need not always be the case. Another embodiment of a device for imaging an interior of a turbid medium is that of a handheld device that may, for instance, be pressed against a side of a turbid medium. In that case, the measurement volume is the volume occupied by the part of the turbid medium from which light is detected as a result of irradiating the turbid medium.

FIG. 2 schematically shows how a device may be arranged to detect light from spatially distinct beams of light emanating from a single exit position. FIG. 2 shows two optical light guides 55 coupled to a single exit position 60 according to an embodiment of the invention. The single exit position 60 may, for instance, come in the form of an optical channel 25b as described in FIG. 1 or it may simply be an area of skin tissue from which light emanates. Light guides have maximum acceptance angles θ within which light must reach the entry openings for light of those light guides in order to enter the entry openings. In FIG. 2 the distance d between the entry openings for light 65 of optical light guides 55 and the width w of the single exit position 60 correspond to the acceptance angles θ such that the tangent of θ is given by the quotient of w/2 and d, for according to the equation: tan(θ)=w/2d. The optical light guides 55 in FIG. 2 are arranged such that both have the same field of view as indicated by the dashed lines. In this way both optical light guides 55 are coupled to the same single exit position 60 as a result of which the light both optical light guides 55 collect contains the same information. Simultaneously collecting light from multiple, spatially distinct light beams reduces measurement times compared to the situation in which light from only one light beam is collected so that measurements involving different detection scheme have to be carried out one after the other. Alternatively, collecting light from multiple, spatially distinct light beams may be used to increase in the signal to noise ratio. Increasing the signal to noise ratio can be achieved by collecting light from multiple, spatially distinct light beams, in a way as illustrated in FIG. 2. The number of signals obtained over a specific period of time for a single exit position 60 is increased as compared to the situation in which light from only one light beam is collected over a same period of time, for instance, by coupling one optical light guide to a single exit location. This embodiment has the advantage that it is easy to implement in the known device, as the known device already comprises single exit positions optically coupled to single optical light guides. In the known device 1 the measurement volume 15 is bound by the receptacle 20. Light guides 30b are optically coupled to the optical channels 25b by inserting the light guides 30b into the optical channels 25b with the ends of the light guides 30b being surrounded by plugs. These plugs can be adapted to enable the optical coupling of multiple light guides 30b to a single optical channel 25b. Satisfying the equation tan(θ)=w/2d may be accomplished by positioning a plug at the correct depth in an optical channel 25b.

FIG. 3 schematically shows two different detection schemes applied to two spatially distinct light beams. The two spatially distinct light beams are indicated by the dashed lines. Two photodetectors, photodetector 70 and photodetector 72, are optically coupled to a single exit position 60. One spatially distinct light beam passes through an optical filter 75, for instance, a fluorescence filter before being detected by the photodetector 70. The other spatially distinct light beam does not pass through an optical filter and is directly coupled to the photodetector 72. FIG. 3 illustrates how various detection schemes may be applied to various spatially distinct light beams. FIG. 3 also illustrates that the optical light guides 55 present in FIG. 2 need not always be present as spatially distinct light beams may be coupled directly to, for instance, photodetectors 70 and 72. Simultaneously applying different detection schemes to multiple, spatially distinct light beams reduces measurement times compared to the situation in which light from only one light beam is collected so that measurements involving different detection scheme have to be carried out one after the other.

FIG. 4 schematically shows means for focusing a light beam emanating from a single exit position. As FIG. 2, FIG. 4 schematically shows two optical light guides 55 optically coupled to a single exit position 60. However, in FIG. 4 lenses 80 are used to focus the spatially distinct light beams that are coupled into the optical light guides 55. The spatially distinct light beams are indicated by the dashed lines. The advantage of focusing at least one light beam emanating from the single exit position 60 is that it enables the implementation of interference filters, since such filters require a focused beam to function properly.

FIG. 5 shows an embodiment of a medical image acquisition device according to the invention. Shown inside the dashed square is essentially the device 1 shown in FIG. 1. However, the light guides 30b shown in FIG. 1 have been replaced by pairs of light guides 55 shown in FIG. 2 according to the invention. The medical image acquisition device 85 shown in FIG. 5 further comprises a screen 95 for displaying a reconstructed image of an interior of the turbid medium 45 and an input interface 100, for instance, a keyboard enabling an operator to interact with the medical image acquisition device 85.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A device (1) for imaging an interior of a turbid medium (45) comprising:
a) a measurement volume (15) for receiving the turbid medium (45);
b) a light source (5) for irradiating the turbid medium (45);
c) a photodetector unit (10) for detecting light emanating from exit positions (60) located on a boundary of the measurement volume (15) as a result of irradiating the turbid medium (45),
characterized in that
the device (1) is arranged to collect light from spatially distinct beams of light emanating from a single exit position (60).

2. A device (1) as claimed in claim 1, the device (1) comprising a plurality of optical light guides (55) having entry openings for light and having acceptance angles for light to enter the entry openings for light and being arranged such that the plurality of optical light guides (55) is optically coupled to the single exit position (60) such that the distances between the entry openings for light of the optical light guides (55) and the single exit position (60) correspond to the acceptance angles of the optical light guides (55).

3. A device (1) as claimed in claim 1, the device (1) being arranged to apply at least two different detection schemes to at least two spatially distinct light beams emanating from a single exit position (60).

4. A device (1) as claimed in claim 1, the device (1) comprising means for focusing at least one light beam emanating from a single exit position (60).

5. A medical image acquisition device (85) comprising:
a) a measurement volume (15) for receiving a turbid medium (45);
b) a light source (5) for irradiating the turbid medium (45);
c) a photodetector unit (10) for detecting light emanating from exit positions (60) located on a boundary of the measurement volume (15) as result of irradiating the turbid medium (45),
characterized in that
the medical image acquisition device (85) is arranged to collect light from spatially distinct beams of light emanating from a single exit position (60).
